(19) United States
(12) Patent Application Publication
(10) Pub. No.: US 2023/0053031 A1
(43) Pub. Date: Feb. 16, 2023

(54) SELF-SANITIZING ENCLOSURE AND METHOD OF OPERATION

(71) Applicant: Panduit Corp., Tinley Park, IL (US)

(72) Inventors: David Shamir, San Jose, CA (US); Samantha Caldera, Midlothian, IL (US)

(73) Assignee: Panduit Corp., Tinley Park, IL (US)

(21) Appl. No.: 17/859,185

(22) Filed: Jul. 7, 2022

Related U.S. Application Data
(60) Provisional application No. 63/260,148, filed on Aug. 11, 2021.

(51) Int. Cl. A61L 2/10; A61L 2/24; A61L 2/28

(52) U.S. Cl. CPC A61L 2/10; A61L 2/24; A61L 2/28; A61L 2202/11; A61L 2202/121; A61L 2202/122; A61L 2202/14

(57) ABSTRACT

Disclosed is a self-sanitizing enclosure for installation onto a table-top. The self-sanitizing enclosure includes a UVC light source, where the UVC radiation emitted from the UVC light source is configured to sterilize components that are stored within the self-sanitizing enclosure.

### CROSS-REFERENCE TO RELATED APPLICATION(S)

This application claims benefit to U.S. Provisional Patent Application No. 63/260,148, filed on Aug. 11, 2021, the entirety of which is hereby incorporated by reference herein.

### FIELD OF THE TECHNOLOGY

This disclosure relates to an in-table enclosure that includes an ultraviolet-C light emitting source for sanitizing components (e.g., cables) that are stored, either partially or fully, within the enclosure.

### BACKGROUND

In order to promote more sterile environments that are free from unwanted germs, efforts are being made to eliminate germs from surfaces of components that may be touched by multiple people. By eliminating the germs from touched surfaces, the potential to physically spread germs is reduced, if not eliminated. While some of the germ eliminating solutions themselves require physical cleaning, other solutions exist for utilizing touchless germ eliminating technology.

The present disclosure will describe a touchless germ eliminating technology for eliminating germs from the inside of an enclosure made for storing components that may be touched by multiple different people.

### SUMMARY

This disclosure relates to a self-sanitizing enclosure for storing one or more cables. The enclosure is configured for installation into a table-top and has been updated to include an ultraviolet C (UVC) light emitting source for self-sanitizing the contents that are stored within the enclosure.

According to an embodiment, a self-sanitizing enclosure is disclosed. The self-sanitizing enclosure includes a top door, a compartment housing including UVC light radiating source configured to radiate UVC light into an internal compartment housing, and a retention nut configured to receive the compartment housing and secure to a structure.

According to an embodiment, a self-sanitizing enclosure is disclosed. The self-sanitizing enclosure including a top door including a visual status indicator and an automatic lock, a compartment housing including a UVC light radiating source configured to radiate UVC light into an internal compartment housing, a memory configured to store computer-executable instructions, and a processor in communication with the memory. The processor is configured to execute the computer-executable instructions to receive a door lock command, control the automatic lock to lock the top door to the compartment housing, control the visual status indicator to present a door locked status, receive a sanitize command, control the UVC light emitting source to radiate, control the visual status indicator to present a sanitizing in-progress status, confirm sanitizing process completion, control the automatic lock to unlock, and control the visual status indicator to present a sanitizing completion status.

### BRIEF DESCRIPTION OF THE DRAWINGS

FIG. 1 is an exploded view of a self-sanitizing enclosure, according to an embodiment.

FIG. 2 is a perspective view of the self-sanitizing enclosure shown in FIG. 1, where the self-sanitizing enclosure is shown in an exemplary installed state being installed onto a table-top.

FIG. 3 is flow chart describing an exemplary process for operating the self-sanitizing enclosure.

FIG. 4 is a system diagram showing an exemplary embodiment of a computer architecture for computing elements of the self-sanitizing enclosure.

### DETAILED DESCRIPTION

This disclosure describes embodiments of a self-sanitizing enclosure for installation onto a table-top. The self-sanitizing enclosure includes a UVC light source, where the UVC radiation emitted from the UVC light source is configured to sterilize components that are stored within the self-sanitizing enclosure. Although the following description provides an exemplary embodiment where the self-sanitizing enclosure is configured to store cables, in other embodiments the self-sanitizing enclosure may be configured to store other components in addition, or in place, of the cables.

The UVC light source may be an LED type light source that emits UVC radiation for disinfecting the air and surfaces it reaches within an inner housing of a compartment housing as described in more detail below. To better aid the UVC radiation in sanitizing the surface of the inner housing, the inner housing of the compartment housing may be a non-porous surface. The UVC light radiation is characterized by having a wavelength between 100-280 nm, and thus is a relatively short waved UV light compared to UVA and UVB types of UV light radiation.

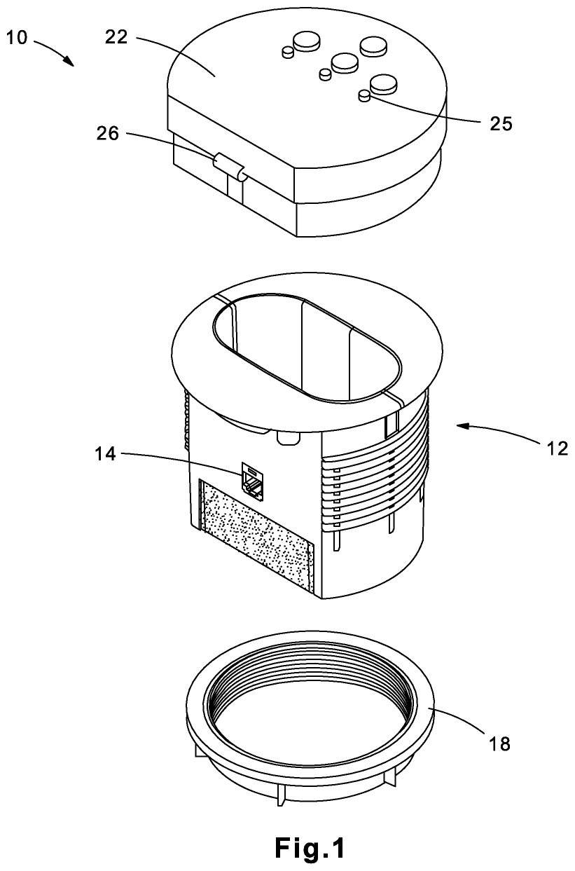

FIG. 1 illustrates an exemplary self-sanitizing enclosure 10 that includes a top door 22, a compartment housing 12, and a retention nut 18. The top door 22 includes one or more visual indicators 25 for providing a visual indication (e.g., color coded) of various states and/or statuses of the self-sanitizing enclosure 10. For example, a first visual indicator may be controlled to indicate the top door 22 being in an opened or closed state, a second visual indicator may be controlled to indicate the top door 22 being in a locked or unlocked state, and a third visual indicator may be controlled to indicate the self-sanitizing enclosure 10 being in a sanitizing in-progress or sanitizing complete state. One or more of the visual indicators 25 may also be controlled to indicate either an on/off state or an in use/not in use state.

The top door 22 further includes a hinge 26 and abase plate, where the base plate is secured to the compartment housing 12. When the base plate is secured to the compartment housing, the hinge 26 is configured to enable rotation about the hinge 26 to open up the top door 22 to expose an internal housing of the compartment housing 12. The internal housing is a space where components, such as cables, may be stored.

Although not shown, the top door 22 also include a door locking mechanism that may be controlled to lock and unlock the top door 22 automatically without human involvement based on a received command. The top door 22 also includes door control mechanism for opening and closing the top door 22 automatically without human involvement based on a received command. The door control mechanism may include a motor for opening and closing the top door 22 about the hinge 26. The automatic locking mechanism and door control mechanism may be included in the self-sanitizing enclosure 10 to reduce a number of human touches to the self-sanitizing enclosure 10, thus further reducing likelihood of contamination.

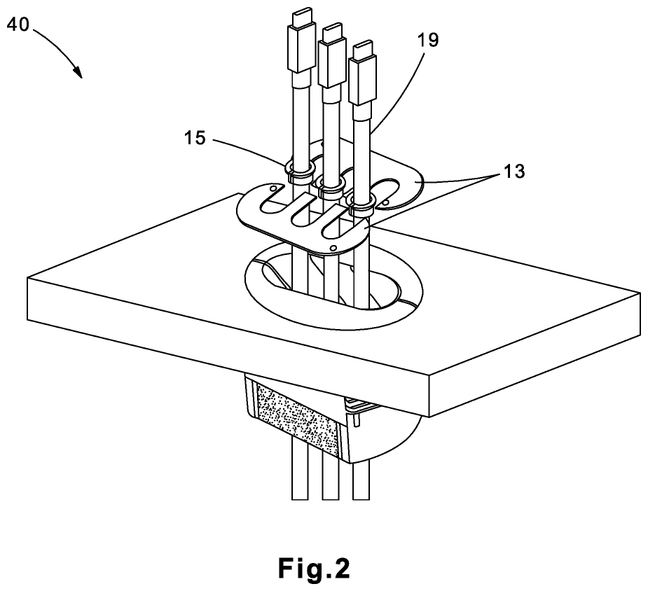

As shown in FIG. 2, the compartment housing 12 may store cables 19. For example, when the compartment housing 12 is configured to store cables, the self-sanitizing enclosure 10 may be an audio-visual (AV) access point installed onto a table-top for use in an enterprise conference room. FIG. 2 further shows the self-sanitizing enclosure 10 including cable slot panels 13 (e.g., come in pairs) and cable-retention washers 15 that work to hold one or more cables 19 within the internal housing of the compartment housing 12. In this way, the cable slot panels 13 work to organize the cables 19 that are stored by the self-sanitizing enclosure 10. Although FIG. 2 shows the cable slot panels 13 including three holding forms for holding the three cables 19, other embodiments may have the cable slot panels 13 including a greater, or lesser, number of holding forms for holding a greater, or lesser, number of cables 19. For example, the cable slot panels 13 may be formed to hold a greater, or lesser, number of cables 19 based on the overall size of the self-sanitizing enclosure 10 that is made for various different installation use cases. It follows that a larger sized self-sanitizing enclosure 10 may be capable of holding a greater number of cables 19, while a smaller sized self-sanitizing enclosure 10 may reduce the number of cables 19 that can be physically stored by the self-sanitizing enclosure 10.

Referring back to FIG. 1, an outer surface of the compartment housing 12 includes threads for engaging with threads on an inner surface of the retention nut 18. So after the retention nut 18 is installed onto a structure (e.g., installed to be relatively flush with a top surface of a table-top), the compartment housing 12 may be rotated about its threads into the threads of the retention nut 18. While this is an exemplary embodiment for how the compartment housing 12 may be secured to the retention nut 18, other embodiments offering different solutions are within the scope of this disclosure. For example, the compartment housing 12 may include latches for snap fitting into latch holes found on the retention nut 18, or the compartment housing 12 may simply be configured to be pressure fit into the retention nut 18.

FIG. 1 also shows a power port 14 for supplying power to the UVC light source and other powered components of the self-sanitizing enclosure 10. The power port 14 may be a power over ethernet (PoE) 802.11.af port configured to provide both power and data via the PoE RJ45 style port.

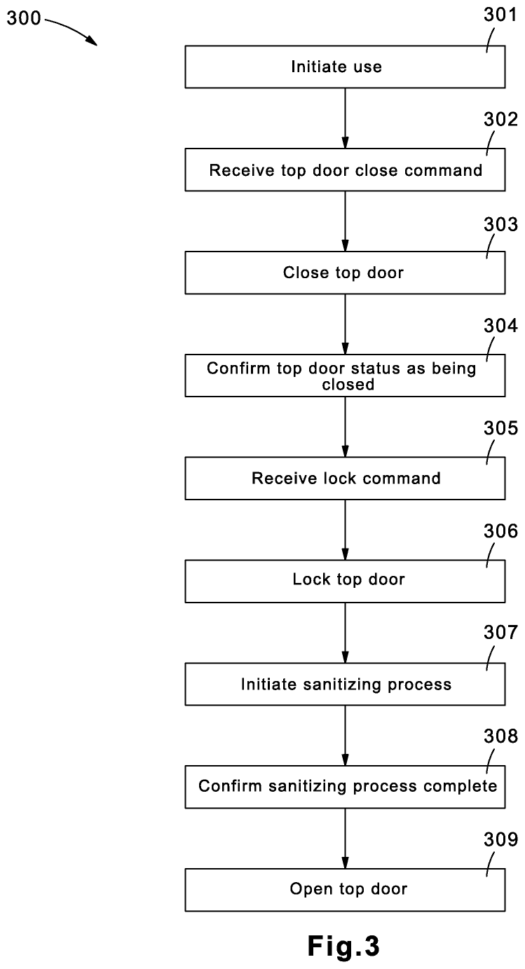

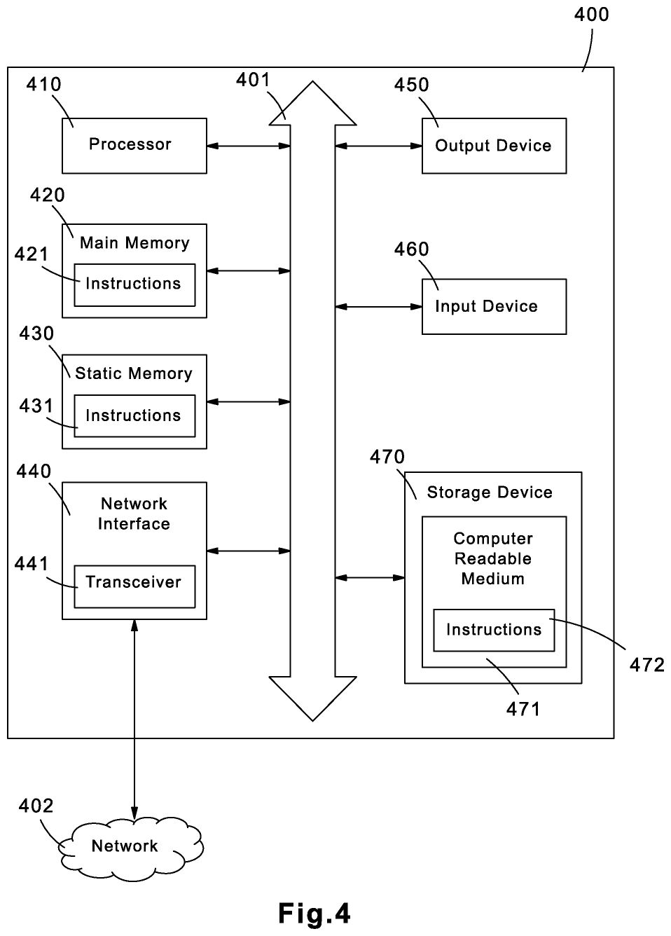

FIG. 3 shows a flow chart 300 describing a process for operating the self-sanitizing enclosure 10. As will be described in more detail with reference to FIG. 4, the self-sanitizing enclosure 10 includes the software, hardware, and circuitry for implementing the features attributed to it as described herein.

At 301, use of the self-sanitizing enclosure 10 is initiated. The initiation may be accomplished when the self-sanitizing enclosure 10 detects one or more cables 19 are being stored within the internal housing of the compartment housing 12. The cables 19 may be detected based on a weighted sensor being triggered by the weight of the cables, an image sensor configured to identify the cables 19, or a proximity sensor that detects when the cables 19 are within close proximity. Upon the initiation of the self-sanitizing enclosure 10, a visual indicator on the top door 22 may indicate that the self-sanitizing enclosure 10 is in an in-use state.

At 302, the self-sanitizing enclosure 10 receives a top door close command for closing the top door. The command may be input remotely by a user that inputs the command from the user's computing device, and received by a data communications interface of the self-sanitizing enclosure 10. The user's computing device may be a personal computer, laptop, smartphone, tablet, in-room control interface, or other computing system configured to receive the command and communicate the command to the self-sanitizing enclosure 10.

At 303, the top door close command is used to control the door control mechanism to close the top door. Then at 304, the self-sanitizing enclosure 10 implements a process for confirming the top door 22 has been successfully shut. Upon confirming the top door 22 has been shut, a visual indicator on the top door 22 may indicate that the top door 22 has been successfully shut. If the visual indicator does not indicate the top door 22 has been successfully shut, a user may intervene to manually shut the top door 22. Alternatively, when the self-sanitizing enclosure 10 cannot confirm that the top door 22 has been shut successfully following the attempt to close the top door 22, the self-sanitizing enclosure 10 may attempt to open the top door 22 and close the top door 22 once again. For example, the self-sanitizing enclosure 10 may repeat the opening and closing of the top door 22 when it cannot confirm successful closing of the top door 22 following a predetermined amount of time passing from initiating the top door 22 closing action.

At 305, the self-sanitizing enclosure 10 receives a lock command for activating the locking mechanism to lock the top door 22 after it has closed. The command may be input remotely by a user that inputs the command from the user's computing device, and received by a data communications interface of the self-sanitizing enclosure 10.

At 306, the self-sanitizing enclosure 10 locks the top door 22 using the locking mechanism. After confirming the top door 22 has been locked, a visual indicator on the top door 22 may indicate that the top door 22 has been locked.

At 307, sanitizing process is initiated. The sanitizing process includes activating the UVC light source to radiate the inner housing of the compartment housing 12 to sanitize the cables 19 that are stored within the compartment housing 12. According to some embodiment, the sanitizing process may be initiated automatically following the confirmed locking of the top door 22. According to other embodiments, the sanitizing process may be initiated based on a sanitizing process initiation command, where the command may be input remotely by a user that inputs the command from the user's computing device, and received by a data communications interface of the self-sanitizing enclosure 10. While the sanitizing process is on-going, a visual indicator on the top door 22 may indicate that the sanitizing process is in progress.

At 308, when the sanitizing process is complete the self-sanitizing enclosure confirms completion. Upon confirming the sanitizing process is complete, a visual indicator on the top door 22 may indicate the sanitizing process is complete.

At 309, the top door 22 may be automatically released upon confirming the completion of the sanitizing process. The top door 22 release may include automatically unlocking the top door 22 using the locking mechanism, and automatically opening the top door 22 using the door control mechanism.

The operating process describe by flow chart 300 is provided for exemplary purposes, as otRO be implemented by the self-sanitizing enclosure 10 that includes additional steps, or removes described steps, or implements steps in different orders.

FIG. 4 illustrates an exemplary computer architecture for a computing device system 400 included in the self-sanitizing enclosure 10, or alternatively, in remote communication with the self-sanitizing enclosure 10. Although not specifically illustrated, the computing device system 400 may additionally include software, hardware, and/or circuitry for implementing attributed features as described herein.

The computing device system 400 includes a processor 410, a main memory 420, a static memory 430, an output device 450 (e.g., a display or speaker), an input device 460, and a storage device 470, communicating via a bus 401. The bus 401 may represent one or more busses, e.g., USB, PCI, ISA (Industry Standard Architecture), X-Bus, EISA (Extended Industry Standard Architecture), or any other appropriate bus and/or bridge (also called a bus controller).

The processor 410 represents a central processing unit of any type of architecture, such as a CISC (Complex Instruction Set Computing), RISC (Reduced Instruction Set Computing), VLIW (Very Long Instruction Word), or a hybrid architecture, although any appropriate processor may be used. The processor 410 may further be a microprocessor. The processor 410 executes instructions 421, 431, 472 stored on one or more of the main memory 420, static memory 430, or storage device 470, respectively. The processor 410 may also include portions of the computing device system 400 that control the operation of the entire computing device system 400. The processor 410 may also represent a controller that organizes data and program storage in memory and transfers data and other information between the various parts of the computing device system 400.

The processor 410 is configured to receive input data and/or user commands through input device 460 or received from a network 402 through a network interface 440. Input device 460 may be a keyboard, mouse or other pointing device, trackball, scroll, button, touchpad, touch screen, keypad, microphone, speech recognition device, video recognition device, accelerometer, gyroscope, global positioning system (GPS) transceiver, or any other appropriate mechanism for the user to input data to computing device system 400 and control operation of computing device system 400 such as user input buttons on the self-sanitizing enclosure 10.

The processor 410 may also communicate with other computer systems via the network 402 to receive control commands or instructions 421, 431, 472, where processor 410 may control the storage of such control commands or instructions 421, 431, 472 into any one or more of the main memory 420 (e.g., random access memory (RAM)), static memory 430 (e.g., read only memory (ROM)), or the storage device 470. The processor 410 may then read and execute the instructions 421, 431, 472 from any one or more of the main memory 420, static memory 430, or storage device 470. The instructions 421, 431, 472 may also be stored onto any one or more of the main memory 420, static memory 430, or storage device 470 through other sources. The instructions 421, 431, 472 may correspond to, for example, instructions for implementing the operation of the self-sanitizing enclosure 10, including the sanitizing process described herein.

Although the computing device system 400 is represented in FIG. 4 as a single processor 410 and a single bus 401, the disclosed embodiments apply equally to computing device systems that may have multiple processors and to computing device systems that may have multiple busses with some or all performing different functions in different ways.

The storage device 470 represents one or more mechanisms for storing data. For example, the storage device 470 may include a computer readable medium 471 such as read-only memory (ROM), RAM, non-volatile storage media, optical storage media, flash memory devices, and/or other machine-readable media. In other embodiments, any appropriate type of storage device may be used. Although only one storage device 470 is shown, multiple storage devices and multiple types of storage devices may be present. Further, although the computing device system 400 is drawn to contain the storage device 470, it may be distributed across other computer systems that are in communication with the computing device system 400, such as a server in communication with the computing device system 400. For example, when the computing device system 400 is representative of a mobile device (e.g., smartphone), the storage device 470 may be distributed across to include a cloud storage platform.

The storage device 470 may include a controller (not shown) and a computer readable medium 471 storing instructions 472 capable of being executed by the processor 410 to carry out control for operating the self-sanitizing enclosure 10, as described herein. In another embodiment some, or all, the functions are carried out via hardware in lieu of a processor-based system. In some embodiments, the included controller is a web application browser, but in other embodiments the controller may be a database system, a file system, an electronic mail system, a media manager, an image manager, or may include any other functions capable of accessing data items.

The output device 450 is configured to present information to the user. For example, the output device 450 may be a display such as a liquid crystal display (LCD), a gas or plasma-based flat-panel display, or a traditional cathode-ray tube (CRT) display or other well-known type of display that may, or may not, also include a touch screen capability. Accordingly, the output device 450 may function to display a graphical user interface (GUI), operational information, or messages to the user, as described herein. In other embodiments, the output device 450 may be a speaker configured to output audible information to the user, or a visual indicator (e.g., colored LED light) for providing a visual indication for certain states or statuses of the self-sanitizing enclosure 10. In still other embodiments, any combination of output devices may be represented by the output device 450.

Computing device system 400 also includes the network interface 440 that allows communication with other computers via the network 402, where the network 402 may be any suitable network and may support any appropriate protocol suitable for communication to/from computing device system 400. In an embodiment, the network 402 may support wireless communications. In another embodiment, the network 402 may support hard-wired communications, such as a telephone line or cable (e.g., PoE ethernet). In another embodiment, the network 402 may support the Ethernet IEEE (Institute of Electrical and Electronics Engineers) 802.3x specification. In another embodiment, the network 402 may be the Internet (e.g., the Internet 1 illustrated in FIG. 1) and may support IP (Internet Protocol). In another embodiment, the network 402 may be a LAN or a wide area network (WAN). In another embodiment, the network 402 may be a hotspot service provider network. In another embodiment, network 402 may be an intranet. In another embodiment, the network 402 may be a GPRS (General Packet Radio Service) network. In another embodiment, the network 402 may be any appropriate cellular data network or cell-based radio network technology. In another embodiment, the network 402 may be an IEEE 802.11 wireless network. In another embodiment, the network 402 may be representative of an Internet of Things (IoT) network. In still another embodiment, the network 402 may be any suitable network or combination of networks. Although one network 402 is shown in FIG. 4, the network 402 may be representative of any number of networks (of the same or different types) that may be utilized.

The network interface 440 provides the computing device system 400 with connectivity to the network 402 through any compatible communications protocol. The network interface 440 sends and/or receives data from the network 402 via a wireless or wired transceiver 441. The transceiver 441 may be a cellular frequency, radio frequency (RF), infrared (IR), Bluetooth, or any of a number of known wireless or wired transmission systems capable of communicating with the network 402 or other computer device having some or all of the features of the computing device system 400. The network interface 440 as illustrated in FIG. 4 may be representative of a single network interface card configured to communicate with one or more different data sources. Furthermore, the network interface 440 may be representative of AV related communication ports such as high-definition multimedia interface (HDMI), DisplayPort, or mini DisplayPort (MDP), as well as data communication ports such as ethernet, universal serial bus (USB), power over ethernet (PoE), or single pair ethernet (SPE).

The computing device system 400 may be implemented using any suitable hardware and/or software, such as a personal computer or other electronic computing device. In addition, the computing device system 400 may also be a smartphone, portable computer, laptop, tablet or notebook computer, PDA, appliance, IP telephone, server computer device, AV gateway, MQTT broker, or mainframe computer.

The computing device system 400 as it relates to the self-sanitizing enclosure 10 may include additional, or fewer, components than shown in FIG. 4 according to different embodiments while remaining within the scope of this disclosure.

Furthermore, while the particular embodiments described herein have been shown and described, it will be obvious to those skilled in the art that changes and modifications may be made without departing from the teaching described herein with reference to the self-sanitizing enclosure 10. The matter set forth in the foregoing description and accompanying drawings is offered by way of illustration only and not as limitation. The actual scope of the disclosure is intended to be defined in the following claims when viewed in their proper perspective.

What is claimed is:

1. A self-sanitizing enclosure comprising:
a top door;
a compartment housing including an ultraviolet C (UVC) light radiating source configured to radiate UVC light into an internal compartment housing; and
a retention nut configured to receive the compartment housing and secure to a structure.

2. The self-sanitizing enclosure of claim 1, wherein the top door comprises:
a plurality of visual indicator lights including at least a door closed status indicator, a door lock status indicator, and a sanitizing status indicator.

3. The self-sanitizing enclosure of claim 1, wherein the UVC light radiating source is a light emitting diode (LED).

4. The self-sanitizing enclosure of claim 1, the compartment housing further including a power port for supplying power to the UVC light radiating source.

5. The self-sanitizing enclosure of claim 1, wherein the structure is a table.

6. The self-sanitizing enclosure of claim 1, further comprising:
a cable slot panel secured within the compartment housing; and
a cable-retention washer secured to the cable slot panel, the cable-retention washer configured to hold a cable.

7. The self-sanitizing enclosure of claim 1, wherein the retention nut includes threads and the compartment housing includes threads on an outer surface, and wherein the retention nut is configured to receive the compartment housing via turning the compartment housing relative to the retention nut using the threads of the retention nut and the threads of the compartment housing.

8. The self-sanitizing enclosure of claim 1, the top door further including a hinge attached to the compartment housing for opening the top door about the hinge to expose the internal compartment housing.

9. The self-sanitizing enclosure of claim 1, the top door further including an automatic lock configured to lock the top door closed onto the compartment housing when a locking command is received.

10. A self-sanitizing enclosure comprising:
a top door including a visual status indicator and an automatic lock;
a compartment housing including an ultraviolet C (UVC) light radiating source configured to radiate UVC light into an internal compartment housing;
a memory configured to store computer-executable instructions; and
a processor in communication with the memory, the processor configured to execute the computer-executable instructions to:
receive a door lock command;
control the automatic lock to lock the top door to the compartment housing;
control the visual status indicator to present a door locked status;
receive a sanitize command; and
control the UVC light radiating source to radiate.

11. The self-sanitizing enclosure of claim 10, wherein the processor is further configured to execute the computer-executable instructions to:
control the visual status indicator to present a sanitizing in-progress status;
confirm sanitizing process completion;
control the automatic lock to unlock; and
control the visual status indicator to present a sanitizing completion status.

12. The self-sanitizing enclosure of claim 10, wherein the processor is further configured to execute the computer-executable instructions to:
receive a door open command;
control the automatic lock to unlock and open the top door; and
control the visual status indicator to present a door unlocked status.
